(19) United States
(12) Patent Application Publication
(10) Pub. No.: US 2026/0083368 A1
(43) Pub. Date: Mar. 26, 2026

(54) WEARABLE MENTAL DISORDER AUTOMATIC DIAGNOSIS SYSTEM AND METHOD BASED ON CONTRASTIVE LEARNING

(71) Applicant: Shenzhen University, Shenzhen (CN)

(72) Inventors: Yongpan ZOU, Shenzhen (CN); Kaishun WU, Shenzhen (CN); Yuda ZHENG, Shenzhen (CN)

(73) Assignee: Shenzhen University, Shenzhen (CN)

(21) Appl. No.: 19/020,987

(22) Filed: Jan. 14, 2025

(30) Foreign Application Priority Data
Sep. 26, 2024 (CN) 202411349860.6

(51) Int. Cl. A61B 5/16; A61B 5/00; G16H 40/60; G16H 50/20

(52) U.S. Cl. CPC A61B 5/16; A61B 5/681; G16H 40/60; G16H 50/20

(57) ABSTRACT

Disclosed are a wearable mental disorder automatic diagnosis system and a method based on contrastive learning. The system is realized by a wearable device and includes a data acquisition unit for obtaining multi-modal physiological data of a user; a user registration unit for executing the following steps under the condition that the user is determined to be a new user: fine-tuning a first feature encoder which is pre-trained offline by using the multi-mode physiological data in a self-supervised contrastive learning mode to obtain a second feature encoder; extracting data features from labeled multi-modal physiological data through a second feature encoder; training a personalized classifier using the data features as input to obtain a mental disorder recognition model; and a recognition unit for obtaining recognition results using the mental disorder recognition model when it is determined that the user is not a new user.

### CROSS-REFERENCE TO RELATED APPLICATIONS

The application claims priority to Chinese Patent Application No. 202411349860.6, filed on Sep. 26, 2024, the entire contents of which are incorporated herein by reference.

### TECHNICAL FIELD

The application relates to the technical field of information processing, in particular to a wearable mental disorder automatic diagnosis system and method based on contrastive learning.

### BACKGROUND

For depressive disorders, anxiety disorders, mood and affective disorders and other diseases, current researchers use different modalities of data to recognize mental disorders, such as text information in the form of questionnaires, behavioral information captured by mobile sensing technology and ecological instantaneous assessment, and deep physiological information obtained by special medical equipment. However, these recognition methods have some problems, such as privacy disclosure, high cost of special equipment, difficulty in obtaining data tags, and low real-time performance. In recent years, with the rapid development of wearable devices, a large number of superficial unlabeled physiological information, such as heart rate variability and blood oxygen saturation, can be obtained anytime and anywhere.

In existing technology, there are mainly three categories of solutions for recognition of different mental disorders. The first type is the traditional recognition method using questionnaires or specialized medical equipment, which faces issues such as privacy breaches, high costs, and lack of ubiquity. The second type is to use ubiquitous devices to collect user behavior information for recognizing and evaluating mental disorders. This method generally obtains user activity, sleep status, etc., which has the problem of low real-time performance. The third type is to use self-supervised learning for recognizing mental disorders. This method usually obtains single modal signals from users for pre training, which has problems such as single modality, difficulty in obtaining data labels, and lack of joint recognition of multiple types of mental disorders.

Studies have shown that mental disorders usually cause physiological reactions, resulting in changes in superficial physiological information. The superficial physiological information directly reflects the real-time physiological state of the body, so that the psychological state of the user can be monitored in time. At present, the related technology research of using contrastive learning to recognize mental disorders is developing rapidly. However, the construction of positive and negative sample pairs in this way does not use multi-modal fusion, and these technical studies usually only focus on one disease. Because there are common symptoms between different mental disorders, it is difficult to recognize which disease an individual has.

Through analysis, in the prior technology, the scheme related to mental disorder recognition mainly has the following defects.

Firstly, in the research of mental disorder recognition, only one mental disorder disease is mostly studied, and a plurality of mental disorder diseases are rarely recognized together. Therefore, users can only determine whether they suffer from the mental disorders studied, but cannot determine whether they suffer from other mental disorders, thus affecting the early detection of users and aggravating the condition.

Secondly, special equipment is used for collecting deep physiological information of a user, so that the cost is high, the operation is complex, the method is not ubiquitous, and the requirement of real-time monitoring in daily life cannot be met.

Thirdly, the mobile sensing technology is used to collect user behavior information, the behavior information of the user for a long time is usually collected, and then the result can be obtained after the information is analyzed, the real-time performance is low, and the instantaneous psychological state of the user is difficult to capture.

Lastly, in the current scheme using self-supervised contrastive learning, the used contrastive learning strategy is generally only for one modality of data, the single-modality physiological information is single, and deeper information cannot be obtained, so the recognition performance is poor.

To sum up, there are few studies on the recognition of multi-class mental disorders based on multi-modal feature fusion of physiological data collected by ubiquitous wearable devices at home and abroad. In addition, the existing research requires a large amount of labeled data and does not support user personalization.

### SUMMARY

The application aims to overcome the defects of the prior technology and provide a wearable mental disorder automatic diagnosis method and system based on contrastive learning.

According to a first aspect of the present application, a wearable mental disorder automatic diagnosis system based on contrastive learning is provided. The system is realized by wearable devices and comprises:

a data acquisition unit configured to obtain multi-modal physiological data of a user;

a user registration unit configured to execute the following steps under the condition that the user is determined to be a new user: fine-tuning a first feature encoder which is pre-trained offline by using the multi-mode physiological data in a self-supervised contrastive learning mode to obtain a second feature encoder; extracting data features from labeled multi-modal physiological data through a second feature encoder; and training a personalized classifier using the data features as input to obtain a mental disorder recognition model, which is constructed based on the second feature encoder and the trained personalized classifier; and

a recognition unit configured to obtain recognition results using the mental disorder recognition model when the user is not determined to be a new user.

According to a second aspect of the present application, a wearable mental disorder automatic diagnosis method based on contrastive learning is provided. The method comprises the following steps of:

acquiring multimode physiological data of a user and determining whether the user is a new user;

executing the following substeps under the condition that the user is determined to be a new user: fine-tuning a first feature encoder which is pre-trained offline by using the multi-mode physiological data in a self-supervised contrastive learning mode to obtain a second feature encoder; extracting data features from labeled multi-modal physiological data through the second feature encoder; and training a personalized classifier using the data features as input to obtain a mental disorder recognition model, which is constructed based on the second feature encoder and the trained personalized classifier; and

obtaining recognition results using the mental disorder recognition model when the user is not determined to be a new user.

Compared with the prior art, the wearable mental disorder automatic diagnosis method has the advantages that the wearable mental disorder automatic diagnosis scheme based on contrastive learning and multi-modal physiological information fusion is generally divided into two phases, wherein in the first phase, the feature encoder is pre-trained in a mode of fusion self-supervised contrastive learning by utilizing label-free superficial physiological information of a large number of patients and normal people; and in the second phase, the feature encoder of the first phase is fine-tuned through a very small amount of labeled superficial physiological information, and a personalized model specific to each user is obtained. The present application utilizes ubiquitous wearable devices to collect superficial unlabeled physiological information from users, and integrates contrastive learning to recognize various mental disorders, achieving aims of real-time monitoring, early detection, and intervention.

Other features and advantages of the present application will become apparent from the following detailed description of exemplary embodiments of the application with reference to the figures.

### BRIEF DESCRIPTION OF THE DRAWINGS

The figures, which are incorporated in and constitute a part of the specification, illustrate embodiments of the application and, together with the description, serve to explain the principles of the application. In the drawings:

FIG. 1 illustrates an overall framework diagram of a wearable mental disorder automatic diagnosis scheme based on contrastive learning according to an embodiment of the present application;

FIG. 2 illustrates an overall framework diagram of a wristband wearable device according to an embodiment of the present application;

FIG. 3 illustrates an appearance diagram of a wristband wearable device according to an embodiment of the present application;

FIG. 4 illustrates an internal structure diagram of a wristband wearable device according to an embodiment of the present application;

FIG. 5 illustrates a schematic diagram of a sampling rule use in accordance with one embodiment of the present application;

FIG. 6 illustrates a schematic diagram of a backbone network and multi-modal fusion strategy according to an embodiment of the present application;

FIG. 7 illustrates a flowchart of a new user registration process according to an embodiment of the present application;

FIG. 8 illustrates a schematic diagram of the process of recognizing mental disorders according to an embodiment of the present application; and

FIG. 9 illustrates a schematic diagram of the recognition results of mental disorders according to an embodiment of the present application.

### DETAILED DESCRIPTION OF THE EMBODIMENTS

Various exemplary embodiments of the present application will now be described in detail with reference to the figures. It should be noted that the relative arrangement of parts and steps, the numerical expressions, and the numerical values set forth in these embodiments do not limit the scope of the application unless specifically stated otherwise.

The following description of at least one exemplary embodiment is merely illustrative in nature and is in no way intended to limit the application, its application, or uses.

Techniques, methods, and devices known to those of ordinary skill in the pertinent art may not be discussed in detail, but should be considered part of the specification, where appropriate.

In all instances shown and discussed herein, any particular value is to be construed as merely illustrative and not restrictive. Thus, other examples of example embodiments may have different values.

It should be noted that like reference numbers and letters refer to like items in the following figures, and therefore, once an item is defined in one figure, it needs not be further discussed in subsequent figures.

Overall, the provided wearable mental disorder automatic diagnosis system based on contrastive learning is implemented using wearable devices, including: a data acquisition unit for obtaining multi-modal physiological data of a user; a user registration unit for executing the following steps under the condition that the user is determined to be a new user: fine-tuning a first feature encoder which is pre-trained offline by using the multi-mode physiological data in a self-supervised contrastive learning mode to obtain a second feature encoder; extracting data features from labeled multi-modal physiological data through the second feature encoder; and training a personalized classifier with the data features as input to obtain a mental disorder recognition model, including the second feature encoder and the trained personalized classifier; and a recognition unit for obtaining recognition results using the stored mental disorder recognition model when it is determined that the user is not a new user. Each unit in the system can be realized by adopting a general processor, a special processor or FPGA, a general sensor and the like in combination with software. In addition, the units may be divided in various ways as long as the functions of the present application can be realized.

The present application proposes a multi-modal superficial physiological information fusion system for recognizing mental disorders based on contrastive learning, combined with ubiquitous wearable devices. The system is generally divided into two phases of training process. In the pre-training phase of the first phase, the cloud pre-trains a feature encoder with a large number of superficial unlabeled physiological information of different users in the way of contrastive learning. In the second phase, the user collects unlabeled superficial physiological information anytime and anywhere through the ubiquitous wearable device, which is personalized by the feature encoder pre-trained in the first phase to recognizing whether the user suffers from mental disorders. By maximizing the similarity of positive samples and minimizing the similarity of negative samples, contrastive learning learns high-quality and recognizable feature representations, which makes the model more clearly distinguish the subtle differences between different mental disorders.

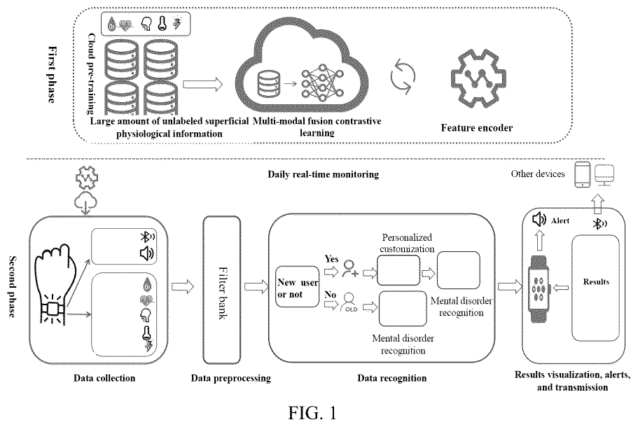

As shown in FIG. 1, the scheme of wearable mental disorder automatic diagnosis based on contrastive learning is divided into two phases. In the first phase, a large number of unlabeled superficial physiological information is collected by smart wearable devices, and a universal feature encoder is pre-trained in the cloud by means of multi-modal fusion contrastive learning, and then the universal feature encoder is deployed to smart wearable devices. In the second phase, when a new user uses the smart wearable device, a small amount of unlabeled superficial physiological information of the new user is first collected, combined with the model parameters and a small amount of data imported before the smart wearable device, and a model suitable for the new user is quickly fine-tuned, so that the personalized model of the new user is customized. Next, the smart wearable device collects the user's superficial physiological information at anytime and anywhere, and sends it to the personalized model after data preprocessing. The user's physiological indicators and the results of recognizing mental disorders can be displayed on the smart wearable device. These data and results can also be transmitted to other devices through wireless transmission protocols (such as Bluetooth protocol) for display. If the user suffers from mental disorders, the voice module of the smart wearable device can also send an alarm.

In one embodiment, a wristband smart wearable device is used, which uses STM32 as the main control board to access various physiological sensors and run system algorithms. Smart wearable devices are equipped with display screens to display recognition results and physiological indicators. This information can also be transmitted to terminal devices through Bluetooth for users to observe intuitively, such as mobile phones or tablet computers.

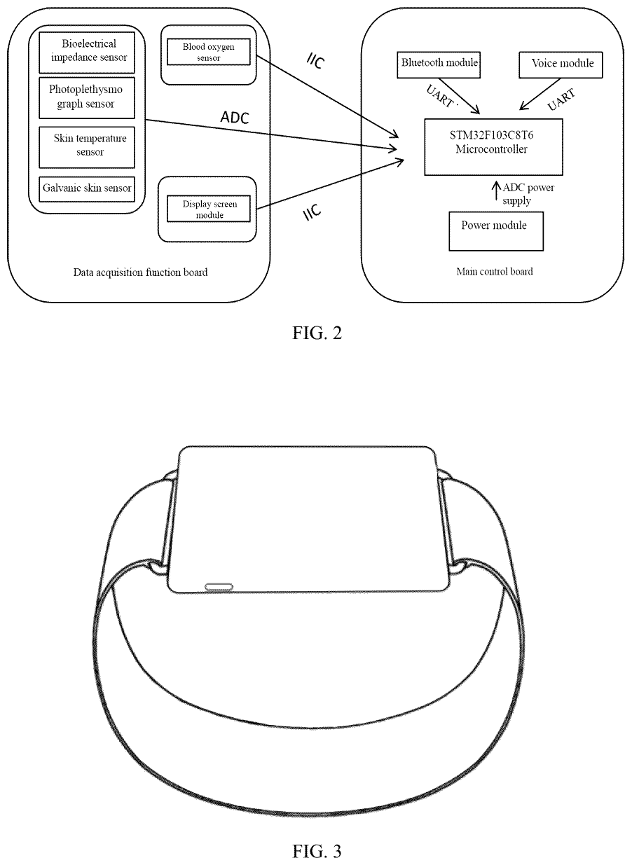

FIG. 2 illustrates an overall framework diagram of the wristband smart wearable device, which generally includes a data acquisition function board and a main control board. The data acquisition function board is provided with a bioelectrical impedance sensor, a blood oxygen sensor, a photoplethysmograph sensor, a galvanic skin sensor, a skin temperature sensor, a display screen module and the like. The main control board is provided with a microcontroller, a voice module, a Bluetooth module, a power supply module and the like. The main control board is in communication connection with each module on the data acquisition function board.

For example, the bioelectrical impedance sensor is connected to the main control board through the analog input pin, and the analog-to-digital converter (ADC) of the main control board reads the value of the respiratory information. The main control board communicates with the blood oxygen sensor through the serial port of the main control board to carry out asynchronous communication so as to read the blood oxygen saturation data. Between the main control board and the photoplethysmograph sensor, the ADC in the main control board is used to read the value of heart rate variability. Between the main control board and the galvanic skin sensor, the ADC in the main control board is used to read the value of skin conductivity change, so as to achieve the purpose of monitoring skin conductance response (EDA). Between the main control board and the skin temperature sensor, an ADC in the main control board is used to read the skin temperature value. Between the main control board and the Bluetooth module, the data of each sensor can be transmitted to the Bluetooth BLE module every 50 ms by using the UART serial protocol, and the Bluetooth BLE module transmits the data to other terminals through the Bluetooth 5.3 protocol, for example. The low-speed serial communication between the main control board and the display screen module is carried out by using the I2C (or called IIC) bus of the main control board, so that the values of each sensor and the processing results are displayed on the display screen. When the main control board receives the data from each sensor and the output result of the model, it sends instructions and data to the voice broadcast module through the UART serial port of the main control board to broadcast the overall situation of the data in the monitoring time period and the final recognition result.

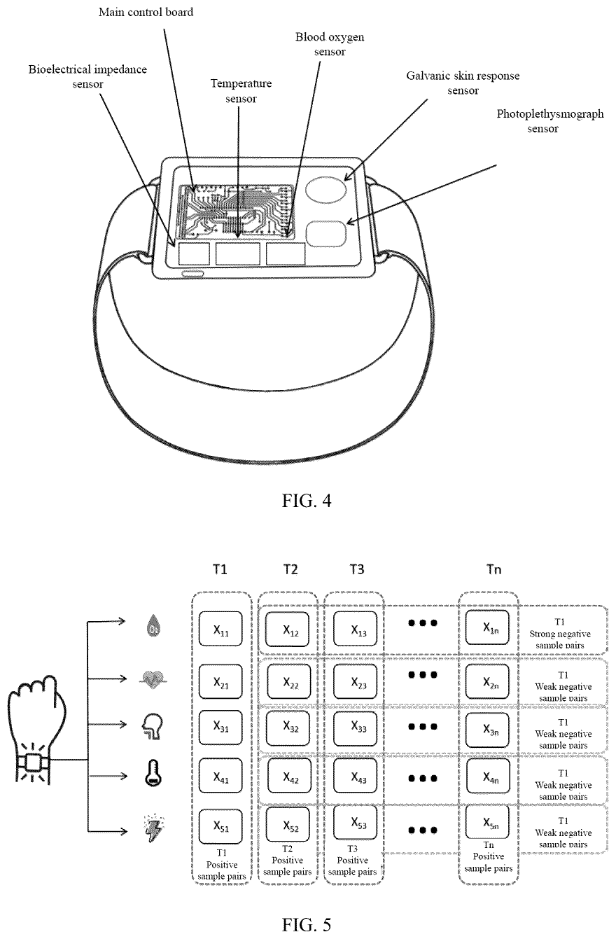

FIG. 3 illustrates an overall appearance view of the wristband-type wearable device, and FIG. 4 illustrates an internal structure view thereof. A display screen is arranged on the top of the equipment and is used for displaying the numerical values, model recognition results and the like of each sensor obtained by the processing of the main control board. Because these sensors need to be close to the skin, the sensors can be embedded in the bottom of the device and close to the wrist of the human body. The Bluetooth module and the voice broadcast module are embedded in the circuit of the main control board.

In the following text, the focus will be on describing the processes of pre training, customizing personalized models, and model application.

### I. Pre-Training Feature Encoder Phase

In daily life, it is easy to obtain the unlabeled physiological data of individuals, but it is difficult to obtain the multi-modal physiological information of individuals with mental disorders, and there are common symptoms among different mental disorders, so it is difficult to recognize which disease an individual suffers from. By maximizing the similarity of positive samples and minimizing the similarity of negative samples, contrastive learning learns high-quality and recognizable feature representations, which makes the model more clearly distinguish the subtle differences between different mental disorders. Contrastive learning uses unlabeled data in the source domain to pre-train the model in a self-supervised way, which can be quickly generalized to different individuals through a small amount of labeled data.

### Positive and Negative Sample Pair Sampling

A key aspect of contrastive learning is the sampling of positive and negative sample pairs. The selection of positive and negative sample pairs in standard contrastive learning is only a coarse-grained division of data into positive and negative sample pairs in the time dimension, but not a fine-grained division of data into positive and negative sample pairs in different modalities of physiological data. In one embodiment of the present application, coarse-grained and fine-grained positive and negative sample pairs are combined to partition multi-modal physiological data, thereby enhancing representation capabilities and enabling more comprehensive feature capture.

FIG. 5 illustrates the sampling rule of the positive and negative samples of the contrastive learning. In the case of time sequence alignment, one modality is selected as the anchor modality at time T1, the physiological data of other modalities at the same time are regarded as positive samples, the samples of the anchor modality at different time points are regarded as strong negative samples, and the samples of other modalities at different time points are regarded as weak negative samples. The ultimate goal of contrastive learning is to maximize the similarity between positive samples and minimize the similarity between negative samples.

For example, coarse-grained sampling can be seen as being implemented in the time dimension of a 1 s sample window, with positive samples being different modes within a 1 s window at the same time, and negative samples being 1 s windows at different times. The fine-grained sampling is implemented on the internal mode of 1 s window, the positive sample is the mutual sampling of different modes at the same time, and the negative sample is divided into two kinds, one is the sampling of the same mode at different times, which is called the strong negative sample, and the other is the sampling of different modes at different times, which is called the weak negative sample. Combining this sampling method with residual variable convolution fusion, coarse-grained sample representation along the time dimension and fine-grained representation across different physiological modalities are achieved. This sampling method significantly improves the accuracy of the recognition results.

### Backbone Network and Multi-Modal Fusion Strategy

Variable convolution can adaptively adjust its sampling position by learning offset, so as to capture local details. When processing time-series data, variable convolution can flexibly adapt to different rhythms and fluctuations in time-series physiological data and capture more accurate time-series characteristics. When fusing multi-modal physiological data, variable convolution can capture the dynamic changes between different modalities or time points in the same modality, and then enhance the effect of feature extraction, so as to fuse data from different modalities more accurately.

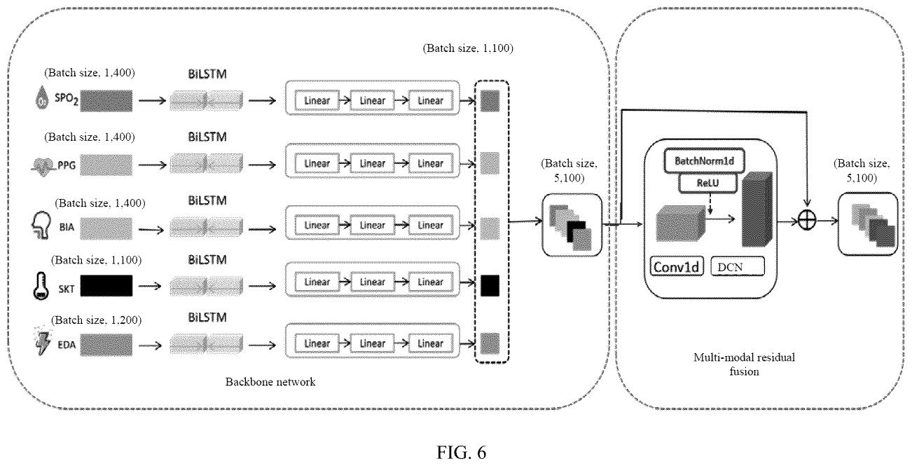

In combination with the above sampling mode, in one embodiment, a residual DCN (Deformable Convolutional Networks) fusion mode is proposed to obtain the local fusion features of the physiological data of different modalities and the global unique features of each modality. FIG. 6 illustrates a schematic diagram of the backbone network and the multi-modal fusion strategy, wherein SPO2 is blood oxygen saturation, BIA is respiration data, EDA is galvanic skin response data, SKT is skin temperature data, and PPG is heart rate variability data. For example, a 1 s window is used as the sample size for selecting physiological information of different modalities. First, each modality passes through different BiLSTM layers to extract the proprietary temporal features of each modality. After passing through the Linear layer, these proprietary features are spliced on the channel dimension to obtain (batch size, 5, 100). Then, the spliced vectors are sent to the residual deformable convolutional network (DCN) to extract the local fusion features and the global unique features of each mode. Different modal physiological data are learned together with coarse and fine granularity, combined with residual DCN fusion, which is conducive to better representing the characteristics of patients and control groups with each disease.

A feature encoder f is pre-trained in the first phase through a large number of unlabeled superficial physiological information processed by the filter bank, and the training can be completed in the cloud or server, thereby saving the computing resources of the smart wearable device.

To sum up, according to the application, the residual variable convolution is used for multi-modal fusion, so that the local fusion feature and the global specific feature of each modality are extracted. The physiological information of different channels is fused together in the form of residual DCN, which not only retains the global information of each channel, but also retains the local information of the fusion mode. Different modal physiological data are learned together with coarse and fine granularity, combined with residual DCN fusion, so that the system can better represent the characteristics of patients with each disease and the control group.

### II. The New User Personalized Customization Model Phase

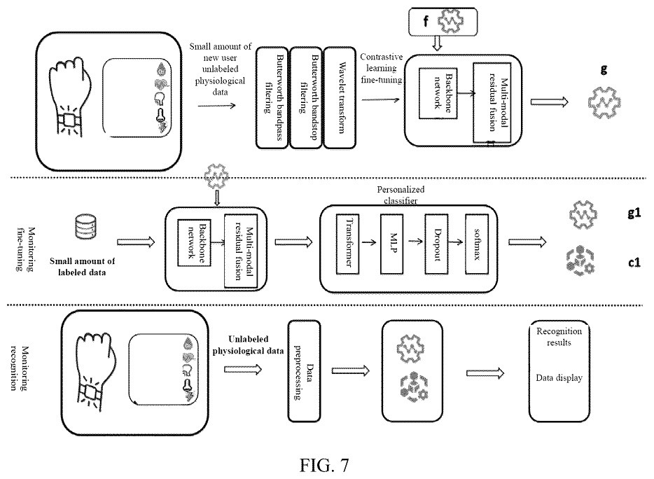

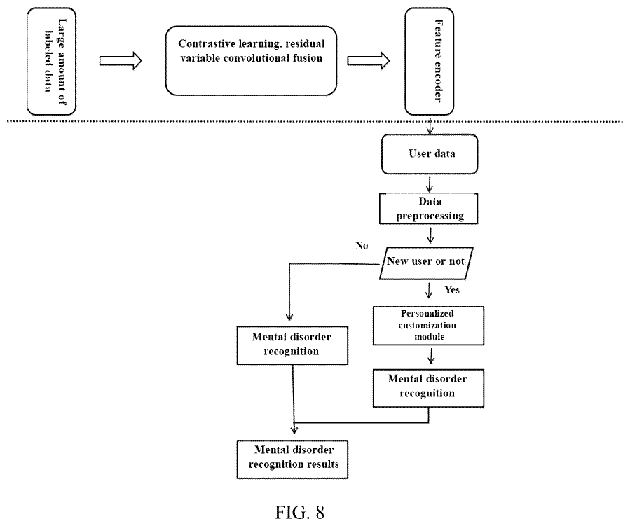

After pre-training the feature encoder, the trained feature encoder and a small amount of labeled multi-modal physiological data are deployed into the wearable device. In daily life scenarios, new users need to register when using the system, so as to obtain personalized customization models belonging to new users. The registration process is shown in FIG. 7. In this process, the wearable device worn by the user dynamically collects multi-modal physiological data for a period of time (such as several minutes), and then the local software preprocesses these data. The processed multi-modal physiological data also fine-tunes the feature encoder f from the pre-training phase in a self-supervised contrastive learning manner. After the fine tuning is completed, a new feature encoder g is obtained. Next, the local software side randomly selects a small amount of multi-modal physiological data with labels in the system, obtains features from the labeled data through an encoder g, and then inputs the features into a classifier consisting of a lightweight transformer, a multilayer perceptron (MLP), a Dropout layer and a Softmax layer. Finally, the feature encoder g1 and the classifier c1 belonging to the new user are customized, so that the new user completes the registration. It can monitor the user's physiological indicators and mental state in real time and determine whether the user suffers from mental disorders.

If the new user keeps wearing the device during the personalized customization process, a conclusion on whether they are sick can be obtained within a few minutes after the customization is completed. If the user is not wearing a device, they need to wear the device to collect multi-modal physiological data for, for example, ten minutes, and then draw a conclusion on whether they are sick.

To sum up, according to the present application, a filter bank combining Butterworth band-pass filtering and band-stop filtering is used to preprocess multi-modal physiological information, and then a deep learning model is used to identify different types of mental disorders, which not only improves the accuracy of recognition, but also saves the resources of wearable devices.

### III. System Recognition of Mental Disorders Process

The system recognizes the process of mental disorders as shown in FIG. 8. Firstly, in the cloud, with the help of a large number of unlabeled multi-modal superficial physiological information processed by the filter bank, a feature encoder is pre-trained in the way of self-supervised contrastive learning, and the feature encoder and a small number of labeled multi-modal shallow physiological information processed by the filter bank are embedded into the smart wearable device. Then, when a user uses the system, it is first determined whether it is a new user, if it is a new user, it is necessary to register the new user, collect the label-free superficial physiological information of the new user through the smart wearable device to fine-tune the feature encoder existing in the device, and then use a small amount of labeled shallow physiological information existing in the device to train a model belonging to the new user. Further, the physiological information of the new user can be monitored and whether the new user suffers from a mental disorder can be recognized; If it is not a new user, the monitoring of the physiological information and the recognition of the presence of a mental disorder are carried out directly.

Through this system, users can know their mental and physiological state anytime and anywhere. The smart wearable device can also send the recognition result and the physiological index information to the mobile phone through the Bluetooth module, so as to better analyze the change of the psychological state and the physiological state of the user; and if the system recognition result is one of the mental disorders, the recognition result can be broadcasted through the voice module, so as to timely remind the user. In this way, mental disorders can be detected and intervened early, thus alleviating psychological and physical symptoms.

Correspondingly, the application also provides a wearable mental disorder automatic diagnosis method based on contrastive learning. The method comprises the following steps of: acquiring multi-mode physiological data of a user and determining whether the user is a new user; executing the following steps under the condition that the user is determined to be a new user: fine-tuning the first feature encoder which is pre-trained offline by using the multi-mode physiological data in a self-supervised contrastive learning mode to obtain a second feature encoder; extracting data features from labeled multi-modal physiological data through the second feature encoder; and training a personalized classifier using the data features as input to obtain a mental disorder recognition model, which is constructed based on the second feature encoder and the trained personalized classifier; and obtaining recognition results using the stored mental disorder recognition model when it is determined that the user is not a new user.

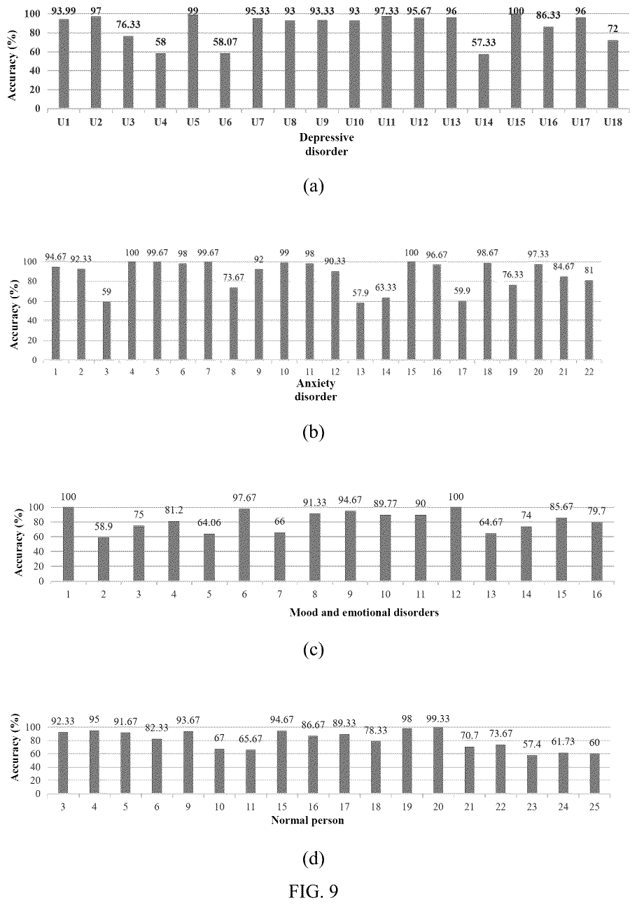

Further, in order to verify the feasibility of the present application, a preliminary experiment was conducted. In the experiment, 18 patients with depressive disorders, 20 patients with anxiety disorders, 16 patients with mood and emotional disorders, and 18 normal people were used, and the test accuracy of each person in each category was obtained by the user leave-one-out method. The results are shown in FIG. 9, wherein (a) is the recognition accuracy rate of depressive disorder, (b) is the recognition accuracy rate of anxiety disorder, (c) is the recognition accuracy rate of mood and affective disorder, and (d) is the recognition accuracy rate of normal person. It can be seen from FIG. 9 that the average recognition accuracy of depressive disorders is 82.51%, the average recognition accuracy of anxiety disorders is 81.97%, the average recognition accuracy of mood and emotional disorders is 82.04%, and the average recognition accuracy of normal people is 80.97%.

It should be noted that those skilled in the art can make appropriate changes or modifications to the above embodiments without departing from the spirit and scope of the present application. For example, various statistical features of the physiological signal, such as mean, variance, standard deviation, median, mode, maximum, minimum, etc., may be used for preprocessing of the data set, and then these statistical features are utilized as a proxy for mental disorder recognition. Alternatively, the physiological signal can be converted into a time-frequency map for processing, and the processed features are transmitted to the system framework for mental disorder recognition. For another example, for the sampling rule of positive and negative sample pairs, the strong and weak negative samples can also be paired together without weighting coefficients, so as to construct negative samples. Bidirectional GRU can also be used instead of BiLSTM. By improving the application, the method is also applicable to the recognition of other types of diseases, such as cardiovascular diseases, as long as the multi-modal physiological data of other types of diseases can be collected.

In summary, the present application has the following advantages over the prior technology.

Firstly, the multi-modal superficial physiological information of the user is collected by means of a ubiquitous and convenient intelligent wearable device. Because mental disorders usually cause physiological reactions that cause changes in superficial physiological information, which directly reflects the immediate physiological state of the body, ubiquitous wearable devices can better meet the real-time requirements of individuals in daily life than special devices and non-physiological acquisition devices, and can detect and intervene in mental disorders early and alleviate psychological and physical symptoms in time.

Secondly, according to the application, a novel contrastive learning mode is combined with residual variable convolution to carry out multi-modal fusion, the similarity between positive samples is maximized through the characteristics of contrastive learning, the similarity between negative samples is minimized, and the residual variable convolution is utilized to carry out multi-modal fusion, so that local fusion features and the global specific features of each modality can be extracted. Different modal physiological data are learned together with coarse and fine granularity, combined with residual DCN fusion, so that the system can better represent the characteristics of patients with each disease and the control group.

Thirdly, the existing self-supervised fine-tuning strategy is to train the classifier of downstream tasks directly with a small amount of labeled data. The present application targets different data for each user. Firstly, unlabeled data is used to fine tune the feature encoder through contrastive learning, and then a small amount of labeled data is used to train classifiers specific to different users, thereby obtaining personalized customized models for each user.

Fourthly, the vast majority of existing work only recognizes one type of mental disorder, with a few works identifying multiple types of mental disorders. However, in terms of the amount of data labels and system performance required, this type of work is not as effective as contrastive learning. The new contrastive learning method adopted in the present application achieves multi-class recognition of mental disorders and can solve the problem of a small number of labels. The trained model has better performance compared to models trained using a large amount of labeled data.

Lastly, there are common symptoms between different mental disorders, it is difficult to recognize which disease an individual has. The present application uses a new contrastive learning positive and negative sample pair construction strategy, which can deeply explore the coarse-grained and fine-grained of various modal physiological data in the entire temporal dimension and at a certain time point, achieving the common recognition of multiple categories of mental disorders (such as depressive disorder, anxiety disorder, and mood and emotional disorders). Compared with existing research, it significantly improves the generalization of the system and can more comprehensively recognize the psychological state of users.

The present application may be a system, method, and/or computer program product. The computer program product may include a computer readable storage medium that is uploaded with computer readable program instructions for causing a processor to implement various aspects of the present application.

A computer readable storage medium may be a tangible device that may hold and store instructions for use by an instruction execution device. A computer readable storage medium may be, for example but not limited to, an electronic storage device, a magnetic storage device, an optical storage device, an electromagnetic storage device, a semiconductor storage device, or any suitable combination of the foregoing. More specific examples (a non-exhaustive list) of the computer readable storage medium would include a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device, such as a punch card or a raised structure in a groove on which instructions are stored, and any suitable combination of the foregoing. The computer readable storage medium used herein is not interpreted as an instantaneous signal itself, such as radio waves or other freely propagating electromagnetic waves, electromagnetic waves propagated through waveguides or other transmission media (e.g. optical pulses through fiber optic cables), or electrical signals transmitted through wires.

The computer readable program instructions described herein can be downloaded to various computing/processing devices from a computer readable storage medium, or to an external computer or external storage device over a network, such as the Internet, a local area network, a wide area network, and/or a wireless network. The network may include copper transmission cables, fiber optic transmission, wireless transmission, routers, firewalls, switches, gateway computers, and/or edge servers. A network adapter card or network interface in each computing/processing device receives computer readable program instructions from the network and forwards the computer-readable program instructions for storage in a computer readable storage medium in the respective computing/processing device.

The computer program instructions used to perform the operations of the present application may be assembly instructions, instruction set architecture (ISA) instructions, machine instructions, machine related instructions, microcode, firmware instructions, state setting data, or source or object code written in any combination of one or more programming languages, including object-oriented programming languages such as Smalltalk, C++, Python, and conventional procedural programming languages such as “C” language or similar programming languages. Computer readable program instructions can be executed entirely on the user's computer, partially on the user's computer, as a standalone software package, partially on the user's computer, partially on a remote computer, or entirely on a remote computer or server. In cases involving remote computers, the remote computer can be connected to the user's computer through any type of network, including a local area network (LAN) or a wide area network (WAN), or can be connected to an external computer (such as using an internet service provider to connect through the internet). In some embodiments, various aspects of the application are implemented by personalizing an electronic circuit, such as a programmable logic circuit, a field programmable gate array (FPGA), or a programmable logic array (PLA), with state information of computer readable program instructions that the electronic circuit may execute.

Various aspects of the present application are described herein with reference to flowchart illustrations and/or block diagrams of methods, apparatus (systems) and computer program products according to embodiments of the application. It should be understood that each box in the flowchart and/or block diagram, as well as combinations of boxes in the flowchart and/or block diagram, can be implemented by computer readable program instructions.

The computer readable program instructions may be provided to a processor of a general purpose computer, special purpose computer, or other programmable data processing apparatus to produce a machine, such that the instructions, when executed by the processor of the computer or other programmable data processing apparatus, resulting in means for implementing the functions/acts specified in the flowchart and/or block diagram block or blocks. The computer readable program instructions may also be stored in a computer readable storage medium that cause a computer, programmable data processing apparatus, and/or other devices to function in a particular manner, such that the computer readable medium storing the instructions comprises an article of manufacture, Which include instructions for implementing various aspects of the functions/acts specified in the flowchart and/or block diagram block or blocks.

The computer readable program instructions may also be loaded onto a computer, other programmable data processing apparatus, or other devices to cause a series of operational steps to be performed on the computer, other programmable data processing apparatus, or other devices, to produce a computer implemented process such that the instructions which execute on the computer, other programmable data processing apparatus, or other devices implement the function/act specified in the flowchart and/or block diagram block or blocks.

The flowchart and block diagrams in the figures illustrate the architecture, functionality, and operation of possible implementations of systems, methods and computer program products according to various embodiments of the present application. In this regard, each block in the flowchart or block diagrams may represent a module, segment, or portion of instructions, which comprise one or more executable instructions for implementing the specified logical function(s). In some alternative implementations, the functions noted in the blocks may occur out of the order noted in the figures. For example, two consecutive blocks may, in fact, be executed substantially concurrently, or they may sometimes be executed in the reverse order, depending upon the functionality involved. It also should be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions. It is well known to those skilled in the art that implementation by means of hardware, implementation by means of software, and implementation by means of a combination of software and hardware are all equivalent.

Various embodiments of the present application have been described above, and the above description is exemplary, not exhaustive, and is not limited to the disclosed embodiments. Many modifications and variations will be apparent to those of ordinary skill in the art without departing from the scope and spirit of the described embodiments. The terminology used in the application was chosen to best explain the principles of the embodiments, the practical application, or technological improvements in the marketplace, or to enable others of ordinary skill in the art to understand the embodiments disclosed herein. The scope of the application is defined by the claims.

What is claimed is:

1. A wearable mental disorder automatic diagnosis system based on contrastive learning, wherein the system is realized by adopting a wearable device and comprises:
a data acquisition unit configured to obtain multi-modal physiological data of a user;
a user registration unit configured to execute the following steps under the condition that the user is determined to be a new user: fine-tuning a first feature encoder which is pre-trained offline by using the multi-mode physiological data in a self-supervised contrastive learning mode to obtain a second feature encoder; extracting data features from labeled multi-modal physiological data through a second feature encoder; and training a personalized classifier using the data features as input to obtain a mental disorder recognition model, which is constructed based on the second feature encoder and the trained personalized classifier; and
a recognition unit configured to obtain recognition results using the mental disorder recognition model when the user is not determined to be a new user.

2. The system according to claim 1, wherein the personalized classifier comprises a transformer network, a multilayer perceptron, a Dropout layer and a Softmax layer.

3. The system according to claim 1, wherein the multi-modal physiological data comprises blood oxygen saturation, respiration information, galvanic skin response, skin temperature, and heart rate variability.

4. The system according to claim 1, wherein in the contrastive learning, a sampling rule of the positive and negative samples is set as follows: for the multi-modal physiological data, in the case of temporal alignment, one modality is selected as an anchor modality at a set time, the physiological data of other modalities at the same time are regarded as positive samples, the sampling of anchor modalities at different time points is regarded as strong negative samples, and the sampling of other modalities at different time points is regarded as weak negative samples.

5. The system according to claim 1, wherein the first feature encoder comprises a plurality of bidirectional long short-term memory networks, a linear layer and a residual deformable convolutional network, each of the bidirectional long short-term memory networks is used to extract a proprietary temporal feature of the physiological data corresponding to one modality, after passing through the linear layer, the special time sequence characteristics of each mode of the physiological data are spliced on the channel dimension to obtain a splicing vector, and the splicing vector is transferred to the residual deformable convolutional network to extract a local fusion feature and a global feature of each mode of the physiological data.

6. The system according to claim 1, wherein an offline pre-training process of the first feature encoder is performed in a cloud or a server, and the unlabeled multi-modal physiological data of different users are used for the offline pre-training in a contrastive learning manner.

7. The system according to claim 1, wherein the multi-modal physiological data monitored by the system and the recognition result of the mental disorder recognition model are transmitted to a terminal device for display through Bluetooth.

8. The system according to claim 3, wherein the system is a wristband wearable device, comprising a main control board, a bioelectrical impedance sensor, a blood oxygen sensor, a photoplethysmograph sensor, a galvanic skin sensor, a skin temperature sensor, a display screen module, a voice broadcast module and a Bluetooth module,
wherein the bioelectrical impedance sensor is connected to the main control board through an analog input pin, and an analog-to-digital converter of the main control board is used for reading the numerical value of respiratory information;
the blood oxygen sensor communicates asynchronously through the serial port of the main control board;
the main control board communicates with the Bluetooth module using universal asynchronous receiver-transmitter (UART) serial port protocol; and
the main control board and the display screen module use I2C bus for serial communication, for sending instructions and data to the voice broadcast module through the UART serial port of the main control board to broadcast physiological data information and the recognition results of the mental disorder recognition model during the monitoring period.

9. A wearable mental disorder automatic diagnosis method based on contrastive learning, comprising the following steps of:
acquiring multimode physiological data of a user and determining whether the user is a new user;
executing the following substeps under the condition that the user is determined to be a new user: fine-tuning a first feature encoder which is pre-trained offline by using the multi-mode physiological data in a self-supervised contrastive learning mode to obtain a second feature encoder; extracting data features from labeled multi-modal physiological data through the second feature encoder; and training a personalized classifier using the data features as input to obtain a mental disorder recognition model, which is constructed based on the second feature encoder and the trained personalized classifier; and
obtaining recognition results using the mental disorder recognition model when the user is not determined to be a new user.

10. A non-transitory computer readable storage medium, having a computer program stored thereon, wherein the computer program, when executed by a processor, implements the steps of the method according to claim 9.
